(19) United States
(12) Patent Application Publication
(10) Pub. No.: US 2026/0240504 A1
(43) Pub. Date: Aug. 20, 2026

(54) ELECTRONIC DEVICE AND METHOD OF TRAINING NEURAL NETWORK FOR SENTIMENT CLASSIFICATION

(71) Applicant: Electronics and Telecommunications Research Institute, Daejeon (KR)

(72) Inventors: Sungpil WOO, Daejeon (KR); YOUNGHO SUH, Daejeon (KR); Jihoon SEO, Daejeon (KR); Sunhwan LIM, Daejeon (KR)

(73) Assignee: Electronics and Telecommunications Research Institute, Daejeon (KR)

(21) Appl. No.: 19/358,621

(22) Filed: Oct. 15, 2025

(30) Foreign Application Priority Data
Feb. 14, 2025 (KR) 10-2025-0019399

(51) Int. Cl. A61B 5/00; A61B 5/0205; A61B 5/16; A61B 5/318; A61B 5/369

(52) U.S. Cl. CPC A61B 5/7267; A61B 5/0205; A61B 5/165; A61B 5/318; A61B 5/369

(57) ABSTRACT

An electronic device and method of training a neural network for sentiment classification are provided. The method includes extracting, based on a plurality of biosignals, features representing an emotional state according to each of the plurality of biosignals, calculating a similarity between the features and a proxy, and analyzing, based on the features and the similarity, contributions indicating a degree to which each of the features contributes to the sentiment classification, and training the neural network to balance the contributions of the features.

### CROSS-REFERENCE TO RELATED APPLICATIONS

This application claims the benefit of Korean Patent Application No. 10-2025-0019399, filed on Feb. 14, 2025, in the Korean Intellectual Property Office, the entire disclosure of which is incorporated herein by reference for all purposes.

### BACKGROUND

### 1. Field of the Invention

One or more embodiments relate to an electronic device and method of training a neural network for sentiment classification.

### 2. Description of the Related Art

Emotional computing (also referred to as affective computing) is a new field of artificial intelligence research and development that concerns designing systems and devices capable of recognizing, interpreting, and processing human emotions. Emotional computing may be defined as computing that relates to, arises from, or influences emotions. Emotional computing is an interdisciplinary field that integrates knowledge from artificial intelligence, cognitive science, and psychology to develop automatic emotion recognition systems and emotion-aware interfaces. The goal is to build powerful computational models for recognizing human emotions and to embed in computers human-like abilities to perceive and interpret emotions. Such affective computing systems may be applied to various industries, including mental health monitoring, safe driving, gaming, and security.

Recently, emotion recognition-based intelligent systems have been applied in diverse areas such as e-health, e-learning, recommender systems, smart homes, smart cities, and intelligent dialogue systems. The use of computer-based automatic emotion recognition has significant potential in various intelligent systems, including online gaming, neuro-marketing (customer feedback evaluation), and mental health monitoring. For example, a medical system equipped with an emotion recognition module may monitor a patient's mental and physical state in real time and prescribe appropriate treatment accordingly. In the field of human-computer interaction (HCI), the goal of emotion recognition and detection may be to design and implement intelligent systems with optimized HCI that may adapt to the emotional state of the user.

Meanwhile, biosignals are related to various human physiological processes. Biosignals may include electroencephalography (EEG), electrocardiography (ECG), electromyography (EMG), galvanic skin response (GSR), body temperature, and respiration (RSP). Biosignals may effectively capture emotion-related information generated in response to external or internal stimuli. Variations in such signals associated with different emotions are induced by the activity of the autonomic nervous system, which controls diverse bodily functions such as heart rate, temperature, pupil response, and digestion. Compared to facial or vocal expressions, one major advantage of biosignals is that they are involuntary and may only be regulated through autonomic activation. For example, the sympathetic and parasympathetic components of the autonomic nervous system may regulate heart rate when activated by internal or external stimuli. Similarly, GSR, EMG, and RSP signals are regulated as a result of sympathetic and parasympathetic nervous activity.

Most emotion recognition systems proposed in prior studies for effective emotion recognition rely on specific sets of features that are artificially designed by developers, which poses a limitation to generalization. In particular, morphological characteristics of biosignals differ across individuals depending on their physiological processes, mental states, and temporal variations. Therefore, to achieve generalization and high performance of emotion classification models, it is necessary to remove reliance on handcrafted features designed by developers. Furthermore, systems designed to operate on a particular type of stimulus data may fail to effectively classify emotions for data originating from other stimuli. Since physiological patterns for similar stimuli may vary significantly across individuals and circumstances, systems may be designed to obtain accurate ground truth of emotions.

When training deep learning models for sentiment classification, conflicts of information among different biosignal modalities (e.g., ECG or EEG) may be one of the major factors degrading the performance of emotion classification models. Each modality has its own unique signal characteristics, and if the interactions among such characteristics are not properly coordinated, conflicts of information among modalities may occur, thereby reducing classification performance. For example, some modalities may provide strong signals for a particular emotional state, whereas others may provide relatively less significant information for that emotional state. In such cases, if the information from important modalities is distorted or the information from less important modalities is excessively reflected, the final classification performance may be negatively affected.

The above description is information the inventor(s) acquired during the course of conceiving the present disclosure, or already possessed at the time, and is not necessarily art publicly known before the present application was filed.

### SUMMARY

Embodiments provide technology for training a neural network to perform sentiment classification by appropriately incorporating interactions among different modalities (e.g., biosignals).

However, technical aspects are not limited to the foregoing aspects, and there may be other technical aspects.

According to an aspect, there is provided a method of training a neural network to perform sentiment classification, the method including extracting, based on a plurality of biosignals, features representing an emotional state according to each of the plurality of biosignals. The method includes calculating a similarity between the features and a proxy. The method includes analyzing, based on the features and the similarity, contributions indicating a degree to which each of the features contributes to the sentiment classification, and training the neural network to balance the contributions of the features. The proxy includes representative features of a class that serves as a criterion for the sentiment classification.

The class may include an arousal class and a valence class.

The plurality of biosignals may include an electroencephalography (EEG) signal and an electrocardiography (ECG) signal.

The features may include a first feature corresponding to the EEG signal and a second feature corresponding to the ECG signal.

The training of the neural network may include calculating, based on the similarity, an attention weight to adjust contributions of the first feature and the second feature. The training of the neural network may include calculating, based on the attention weight, the proxy, and the first feature, a first mean weight based on the first feature. The training of the neural network may include calculating, based on the attention weight, the proxy, and the second feature, a second mean weight based on the second feature. The training of the neural network may include adjusting, based on the first mean weight, the second mean weight, and the similarity, parameters of the neural network.

The adjusting of the parameters of the neural network may include summing the first mean weight and the second mean weight to obtain a summed weight for balancing a contribution of the first feature and a contribution of the second feature. The adjusting of the parameters of the neural network may include calculating, based on the summed weight and the similarity, a loss function for adjusting a balance between the contributions.

The adjusting of the parameters of the neural network may include adjusting the parameters of the neural network so that the loss function is minimized.

According to another aspect, there is provided an electronic device for training a neural network to perform sentiment classification, the electronic device including a processor. The electronic device includes a memory configured to store instructions. The instructions, when executed individually or collectively by the processor, cause the electronic device to extract, based on a plurality of biosignals, features representing an emotional state according to each of the plurality of biosignals. The instructions, when executed individually or collectively by the processor, cause the electronic device to calculate a similarity between the features and a proxy. The instructions, when executed individually or collectively by the processor, cause the electronic device to analyze, based on the features and the similarity, contributions indicating a degree to which each of the features contributes to the sentiment classification, and train the neural network to balance the contributions of the features. The proxy includes representative features of a class that serves as a criterion for sentiment classification.

The class may include an arousal class and a valence class.

The plurality of biosignals may include an EEG signal and an ECG signal.

The features may include a first feature corresponding to the EEG signal and a second feature corresponding to the ECG signal.

The instructions, when executed individually or collectively by the processor, may cause the electronic device to calculate, based on the similarity, an attention weight to adjust contributions of the first feature and the second feature. The instructions, when executed individually or collectively by the processor, may cause the electronic device to calculate, based on the attention weight, the proxy, and the first feature, the first mean weight based on the first feature. The instructions, when executed individually or collectively by the processor, may cause the electronic device to calculate, based on the attention weight, the proxy, and the second feature, the second mean weight based on the second feature. The instructions, when executed individually or collectively by the processor, may cause the electronic device to adjust, based on the first mean weight, the second mean weight, and the similarity, parameters of the neural network.

The instructions, when executed individually or collectively by the processor, may cause the electronic device to sum the first mean weight and the second mean weight to obtain a summed weight for balancing a contribution of the first feature and a contribution of the second feature. The instructions, when executed individually or collectively by the processor, may cause the electronic device to calculate, based on the summed weight and the similarity, a loss function for adjusting a balance between the contributions.

The instructions, when executed individually or collectively by the processor, may cause the electronic device to adjust the parameters of the neural network so that the loss function is minimized.

### BRIEF DESCRIPTION OF THE DRAWINGS

FIG. 1 illustrates an example of a sentiment classification system according to an embodiment.

FIG. 2A is a diagram illustrating a deep learning operation method using an artificial neural network.

FIG. 2B is a diagram illustrating a training and inference method of an artificial neural network model according to an embodiment.

FIG. 3 is a diagram illustrating a sentiment classification framework according to an embodiment.

FIG. 4 is a diagram illustrating a neural network for performing sentiment classification according to an embodiment.

FIG. 5 is a diagram illustrating an intra-modality attention network illustrated in FIG. 4.

FIG. 6 is a diagram illustrating an inter-modality attention network illustrated in FIG. 4.

FIG. 7 is a flowchart illustrating a training method of a neural network according to an embodiment.

FIG. 8 illustrates an example of an electronic device according to an embodiment.

### DETAILED DESCRIPTION

The following detailed structural or functional description is provided as an example only and various alterations and modifications may be made to the examples. Accordingly, the example embodiments are not construed as limited to the disclosure and should be understood to include all changes, equivalents, and replacements within the idea and the technical scope of the disclosure.

Terms, such as “first”, “second”, and the like, may be used herein to describe components. Each of these terminologies is not used to define an essence, order or sequence of a corresponding component but used merely to distinguish the corresponding component from other component(s). For example, a first component may be referred to as a second component, and similarly the second component may also be referred to as the first component.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a”, “an”, and “the” include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms used herein including technical or scientific terms have the same meaning as commonly understood by one of ordinary skill in the art to which examples belong. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like elements and a repeated description related thereto will be omitted.

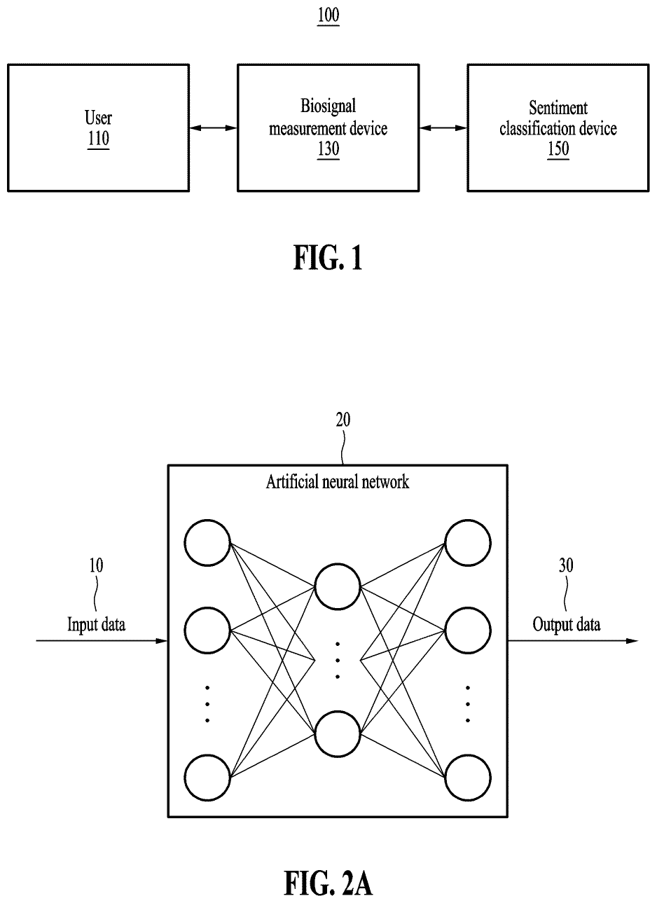

FIG. 1 illustrates an example of a sentiment classification system according to an embodiment.

Referring to FIG. 1, a sentiment classification system 100 may include a biosignal measurement device 130 and a sentiment classification device 150. However, FIG. 1 is provided as an example for explaining the present disclosure, and it should not be construed that the scope of the present disclosure is limited thereto. For example, the biosignal measurement device 130 and the sentiment classification device 150 may be implemented as a single device.

The biosignal measurement device 130 may measure a biosignal of a user 110 through various sensors. A biosignal may include an electroencephalography (EEG) signal and an electrocardiography (ECG) signal.

The biosignal measurement device 130 may transmit the measured biosignal to the sentiment classification device 150. The biosignal measurement device 130 may pre-process the biosignal and transmit the pre-processed biosignal to the sentiment classification device 150. However, the present disclosure is not limited thereto, and the pre-processing of a biosignal may alternatively be performed by the sentiment classification device 150. Hereinafter, the following description is given on the assumption that the pre-processing of a biosignal is performed by the sentiment classification device 150.

The sentiment classification device 150 may obtain (e.g., receive) a biosignal measured by the biosignal measurement device 130. The sentiment classification device 150 may pre-process the biosignal for use in sentiment classification.

With respect to the ECG signal, the sentiment classification device 150 may calculate a heart rate variability (HRV) sequence after detecting an R peak. The sentiment classification device 150 may normalize the ECG signal before calculating the HRV sequence. To uniformly maintain the lengths of all segments of the ECG signal, the sentiment classification device 150 may employ a zero embedding technique, in which 0 is added to an end of each sample so that the lengths of the segments are uniformly maintained.

With respect to the EEG signal, the sentiment classification device 150 may calculate features of each EEG band via differential entropy. The sentiment classification device 150 may divide EEG signals of all channels into four frequency bands (e.g., theta, alpha, beta, and gamma). The sentiment classification device 150 may calculate the differential entropy for each frequency band of all channels according to Equations 1 and 2 below.

$$f ( X ) = {\int }_{- ∞}^{∞} \frac{1}{\sqrt{2 π {σ}^{2}}} exp \frac{{( x - μ )}^{2}}{2 {πσ}^{2}} log \frac{1}{\sqrt{2 π {σ}^{2}}} \qquad [ Equation 1 ]$$

$$exp \frac{{( x - μ )}^{2}}{2 π {σ}^{2}} = \frac{1}{2} log 2 π e {σ}^{2} \qquad [ Equation 2 ]$$

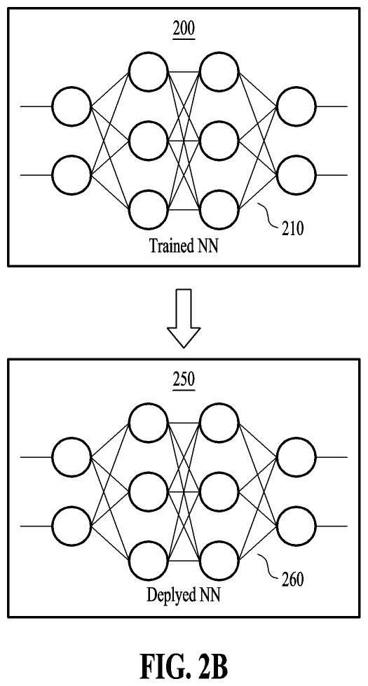

The sentiment classification device 150 may perform sentiment classification by pre-processing a plurality of biosignals (e.g., EEG signals and ECG signals). In this case, when performing sentiment classification based on the plurality of biosignals, it may be important to uniformly reflect a degree (e.g., contribution) to which each biosignal contributes to sentiment classification while considering interactions among the plurality of biosignals. An artificial intelligence (AI) algorithm may be used for sentiment classification. During training of a neural network for sentiment classification, it may be necessary to adjust parameters of the neural network to uniformly reflect the interactions among the plurality of biosignals. Hereinafter, the AI algorithm will first be described with reference to FIGS. 2A and 2B before describing a training method of a neural network according to an embodiment.

FIG. 2A is a diagram illustrating a deep learning operation method using an artificial neural network (ANN).

An AI algorithm, including deep learning, may provide input data 10 as an input into the ANN and train to generate output data 30 through operations such as convolution. The ANN may refer to a computational architecture modeling a biological brain. Within the ANN, nodes corresponding to neurons of the brain are interconnected and collectively operate to process input data. For example, various types of neural networks include a convolutional neural network (CNN), a recurrent neural network (RNN), a deep belief network (DBN), or a restricted Boltzmann machine (RBM), but the present disclosure is not limited thereto. In a feed-forward neural network, neurons of the neural network have links to other neurons, and such links are extended in one direction, e.g., a forward direction, through the neural network.

FIG. 2A illustrates a structure in which the input data 10 is provided to the ANN, and the output data 30 is generated through the ANN (e.g., a CNN 20) including one or more layers. The ANN may be a DNN having two or more layers.

The CNN 20 may be used to extract features such as edges, lines, and colors from the input data 10. The CNN 20 may include a plurality of layers. Each layer may receive input data, process the input data, and generate output data. The output data of a layer may be a feature map generated by performing a convolution operation between an image input to the CNN 20 or a feature map input to the CNN 20 and a weight value of one or more filters. Initial layers of the CNN 20 may operate to extract low-level features such as edges or gradients from the input. Subsequent layers of the CNN 20 may gradually extract more complex features such as eyes or noses within the image.

FIG. 2B is a diagram illustrating a training and inference method of an ANN model according to an embodiment.

Referring to FIG. 2B, a sentiment classification system (e.g., the sentiment classification system 100 of FIG. 1) according to an embodiment may include a training device 200 and an inference device 250. The training device 200 may be a computing device having various processing functions such as generating a neural network, training (or learning) the neural network, or retraining the neural network. For example, the training device 200 may be implemented in various types of devices, such as a personal computer (PC), a server device, or a mobile device.

The training device 200 may generate one or more trained neural networks 210 by repeatedly training a given initial neural network. The generation of the one or more trained neural networks 210 may refer to determining parameters of the neural network. Here, the parameters may include, for example, various types of data such as activations, weights, or biases of the neural network, which are input to or output from the neural network. As the neural network undergoes repeated training, the parameters of the neural network may be tuned to generate a more accurate output with respect to a given input.

The training device 200 may transmit one or more trained neural networks 210 to the inference device 250. The inference device 250 may be included in a mobile device or an embedded device. According to an embodiment, the inference device 250 may be dedicated hardware for operating a neural network and may be an electronic device including at least one of a processor, a memory, an input/output (I/O) interface, a display, a communication interface, and a sensor.

The inference device 250 may encompass all digital devices having an operational capability with a memory and a microprocessor, including a tablet PC, a smartphone, a personal computer (PC) (e.g., a laptop computer), an AI speaker, a smart television (TV), a mobile phone, a navigation device, a web pad, a personal digital assistant (PDA), or a workstation.

The inference device 250 may execute one or more trained neural networks 210 as they are or may execute a processed neural network 260 obtained by processing (e.g., quantizing) the one or more trained neural networks 210. The processed neural network 260 may be implemented in a device independent of the training device 200. However, the present disclosure is not limited thereto. The inference device 250 may also be implemented within the same device as the training device 200. For example, the training device 200 and/or the inference device 250 may be implemented as a sentiment classification device (e.g., the sentiment classification device 150 of FIG. 1).

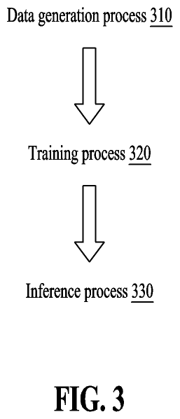

FIG. 3 is a diagram illustrating a sentiment classification framework according to an embodiment.

Referring to FIG. 3, the sentiment classification framework may include a data generation process 310, a training process 320, and an inference process 330.

According to an embodiment, the data generation process 310 may be a process of generating training data required for the training process 320. The data generation process 310 may be performed by a data generation device. According to an embodiment, the data generation device may be implemented by the training device 200 or by a separate device (not shown). For example, the data generation process 310 may be performed by a separate device, and the training device 200 may receive training data generated through the data generation process 310 and perform training using the training data.

The data generation device may generate or collect a training dataset for training an ANN model (e.g., a sampling distribution estimation model).

In the training process 320, the training device 200 may train a neural network model (e.g., a sentiment classification model or the neural network 210 of FIG. 2B) to reflect features of different modalities (e.g., biosignals) in a balanced manner and perform sentiment classification.

In the inference process 330, the inference device 250 may perform sentiment classification while reflecting features of different modalities (e.g., biosignals) in a balanced manner using a trained ANN model (e.g., a trained sentiment classification model or the neural network 210 of FIG. 2B and/or the processed neural network 260 of FIG. 2B).

Hereinafter, a method of training the neural network 210 to perform sentiment classification will be described in detail, particularly with respect to reflecting features of different modalities (e.g., biosignals) in a balanced manner.

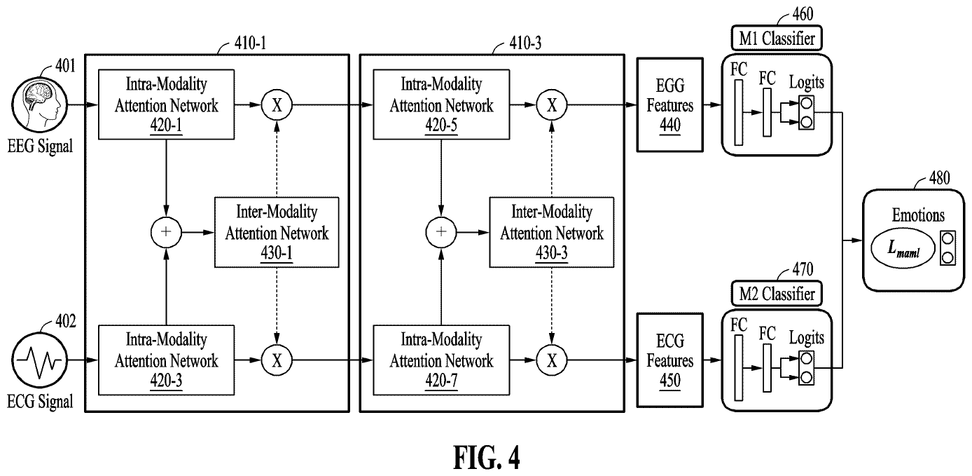

FIG. 4 is a diagram illustrating a neural network for performing sentiment classification according to an embodiment.

Referring to FIG. 4, a neural network 400 (e.g., the neural network 210 of FIG. 2B) may include one or more modality-aware attention networks 410-1 and 410-3, an M1 classifier 460, and an M2 classifier 470. Although FIG. 4 illustrates two modality-aware attention networks 410-1 and 410-3, the present disclosure is not limited thereto. The neural network 400 may include one or three or more modality-aware attention networks.

The modality-aware attention network 410-1 may include intra-modality attention networks 420-1 and 420-3, and an inter-modality attention network 430-1. The intra-modality attention networks 420-1 and 420-3 may correspond to biosignals 401 and 402, respectively. Biosignals 401 and 402 respectively correspond to the EEG signal 401 and the ECG signal 402. When the biosignals further include signals other than the EEG and ECG signals, additional intra-modality attention networks may be added accordingly.

The intra-modality attention network 420-1, based on the EEG signal 401, may extract a feature (e.g., a first feature) related to an emotional state from the EEG signal 401. The intra-modality attention network 420-1 may extract and emphasize an important feature related to the emotional state from the EEG signal 401. The intra-modality attention network 420-1 may analyze a temporal pattern of an EEG signal and extract an important feature from a frequency band. The intra-modality attention network 420-1 may analyze the extracted features in a multi-layer structure and emphasize important features according to a level of abstraction of each layer.

The intra-modality attention network 420-3, based on the ECG signal 402, may also extract a feature (e.g., a second feature) related to an emotional state from the ECG signal 402. Since the operations performed by the intra-modality attention network 420-3 are substantially the same as those performed by the intra-modality attention network 420-1, a repeated description thereof will be omitted hereinafter.

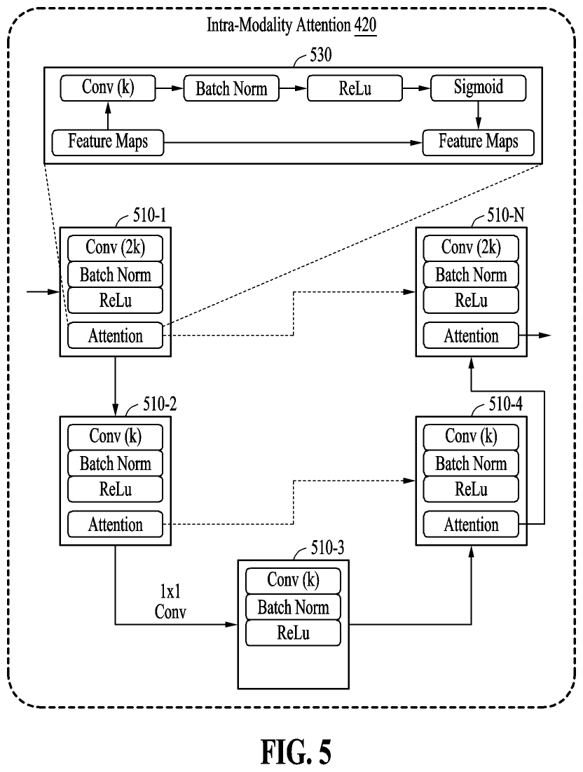

The structure and specific operations of the intra-modality attention networks 420-1 and 420-3 will be described in detail below with reference to FIG. 5.

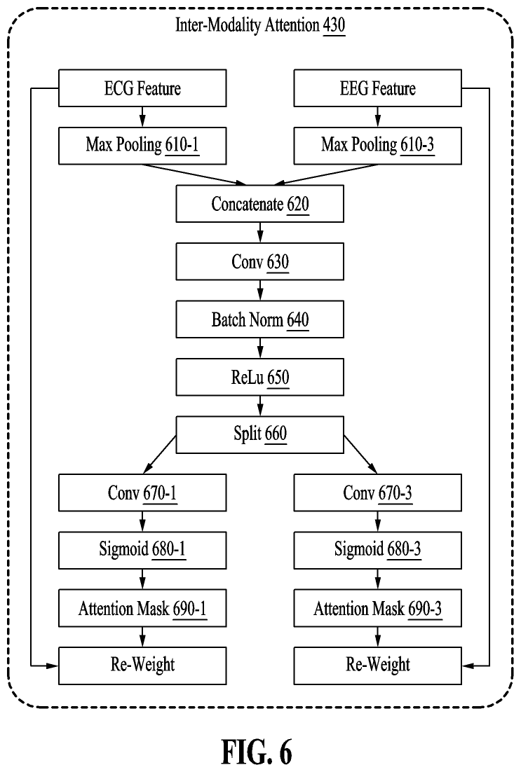

The inter-modality attention network 430-1 may effectively model an interaction between the biosignals 401 and 402 and combine features (e.g., the first feature corresponding to the EEG signal 401 and the second feature corresponding to the ECG signal 402) extracted from the biosignals 401 and 402, thereby improving the performance of sentiment classification. The inter-modality attention network 430-1 may analyze an interaction between the first feature and the second feature, combine the first feature and the second feature, and emphasize an important feature. The structure and specific operations of the intra-modality attention network 430-1 will be described in detail with reference to FIG. 6.

The modality-aware attention network 410-3 may have substantially the same structure as the modality-aware attention network 410-1. The output of the modality-aware attention network 410-1 is input to the modality-aware attention network 410-3, and processes (e.g., extracting features and emphasizing important information) performed in the modality-aware attention network 410-1 may be performed again in substantially the same manner. This may enhance the accuracy of extracting and emphasizing important features by passing through a plurality of networks. Although FIG. 4 illustrates the processes through two modality-aware attention networks 410-1 and 410-3, the present disclosure is not limited thereto.

A training device (e.g., the training device 200 of FIG. 2B) may extract features (e.g., a first feature 440 and a second feature 450) from the biosignals 401 and 402 through the modality-aware attention networks 410-1 and 410-3.

The training device 200 may train the neural network 400 using a proxy-based loss function. The proxy-based loss function may maximize complementary contributions of the EEG signal 401 and the ECG signal 402 and improve classification performance. The proxy-based loss function may enable all the biosignals 401 and 402 to uniformly and complementarily contribute to the training process while maintaining unique characteristics of each of the biosignals 401 and 402. The proxy-based loss function may clearly distinguish between classes and the biosignals 401 and 402 by using a plurality of proxies for each of the biosignals 401 and 402 and their corresponding classes, and may minimize intra-class variance within the classes and the biosignals 401 and 402. Hereinafter, a method of calculating the proxy-based loss function will be described.

Before describing the method of calculating the loss function, the proxies and classes used in the present disclosure will be described.

The proxies may include (or refer to) representative features within a class which serves as a criterion for sentiment classification. The representative features may be weight vectors representing each class. The classes may include an arousal class and a valence class, and may be categories according to a binary sentiment classification. A plurality of proxies may be defined for each class and the biosignals 401 and 402. The proxies may be used to calculate a similarity with features extracted from the corresponding biosignal for each class.

The training device 200 may calculate a similarity between features (e.g., the first feature 440 and the second feature 450) and a proxy. The similarity may be a value that quantifies a relation between a feature (e.g., information or data obtained from the biosignals 401 and 402) and a proxy, and a higher similarity value indicates that the data of a specific biosignal is determined to be more relevant to the corresponding class. The training device 200 may respectively calculate similarities between the features and a plurality of proxies. Accordingly, a similarity between the first feature 440 and the plurality of proxies and a similarity between the second feature 450 and the plurality of proxies may be calculated. For example, the training device 200 may calculate a similarity between a feature and a proxy as shown in Equation 3 below.

$${sim}_{i , c}^{k} = {\sum }_{m} {x}_{i}^{m} \cdot {w}_{c}^{k , m} \qquad [ Equation 3 ]$$

In Equation 3,

$${sim}_{i , c}^{k}$$

denotes a similarity between an ith feature and a kth proxy in class c,

$${x}_{i ,}^{m}$$

denotes an ith feature of a biosignal m, and

$${w}_{c}^{k , m}$$

denotes the kth proxy for class c and the biosignal m. The ith feature denotes an index of a modality-aware attention network and may denote a feature extracted through an ith modality-aware attention network. In other words, if the same biosignal (e.g., the EEG signal 401) is processed n times through the modality-aware attention network, features from the first to the nth may be present.

The training device 200 may normalize the similarity. The normalized similarity may indicate how close a feature of each biosignal is to a specific class. The training device 200 may compare a similarity normalized for a specific class with similarities for all classes, evaluate how much the corresponding biosignal contributes to sentiment classification, and obtain a contribution of the corresponding biosignal. The training device 200 may normalize the similarity through a softmax function. The training device 200 may define the same number of proxies for each biosignal within a class through the softmax function, apply a weight of the proxy, and then perform normalization. In other words, the training device 200 does not simply calculate a softmax of the similarity, but calculates similarities with proxies by each biosignal, normalizes the similarities, and thereby more accurately reflects a correlation between each modality and a class. The training device 200 may, for example, normalize the similarity according to Equation 4 below.

$${S}_{i , c} = {\sum }_{k} \frac{exp ( \frac{1}{γ} {sim}_{i , c}^{k} )}{{\sum }_{c} exp ( \frac{1}{γ} {sim}_{i , c}^{k} )} {sim}_{i , c}^{k} \qquad [ Equation 4 ]$$

In Equation 4, S_{i,c} denotes a similarity normalized for class c, γ denotes a normalization coefficient, which enables contributions of biosignals to be uniformly maintained during a training process. Other parameters may be substantially the same as those of Equation 3.

The training device 200 may calculate an attention weight based on the similarity to adjust contributions of the first feature 440 and the second feature 450. The attention weight may determine which proxy is relatively more important by comparing similarities with a plurality of proxies. The training device 200 may identify an important proxy that has a significant impact on sentiment classification among the plurality of proxies based on the attention weight. The attention weight may be used to adjust the relative contributions of the biosignals 401 and 402. The training device 200 may, for example, calculate the attention weight according to Equation 5 below.

$${att}_{i , c}^{k} = \frac{exp ( \frac{1}{γ} {sim}_{i , c}^{k} )}{{\sum }_{c} {\sum }_{k} exp ( \frac{1}{γ} {sim}_{i , c}^{k} )} \qquad [ Equation 5 ]$$

In Equation 5,

$${att}_{i , c}^{k}$$

denotes the attention weight of the kth proxy for class c. Other parameters may be substantially the same as those of Equations 3 and 4.

The training device 200, based on an attention weight, a proxy, and features, may calculate a mean weight for each feature. The training device 200, based on an attention weight, a proxy, and the first feature 440, may calculate a first mean weight for the first feature 440. The training device 200, based on an attention weight, a proxy, and the second feature 450, may calculate a second mean weight for the second feature 450. The training device 200 may calculate a mean weight for features by adjusting contributions of the features through an attention weight. The training device 200 may calculate a mean weight of a specific biosignal for a class by incorporating data from a plurality of classes and a plurality of proxies for each class within the same biosignal. The training device 200 may calculate weights of features extracted through all modality-aware attention networks 410-1 and 410-3 by each biosignal and calculate an average (e.g., a mean weight) of the calculated weights. The reason for using a mean weight is to evaluate contributions by reflecting biosignal information that reacts significantly across all layers, since some information may react significantly only at a specific layer. The training device 200, for example, may calculate a mean weight according to Equations 6 and 7 below.

$${A}_{i} = {\sum }_{m} ( {\sum }_{c} {\sum }_{k} {att}_{i , c}^{k} \cdot {w}_{c}^{k , m} + J ) ⊗ {x}_{i}^{m} \qquad [ Equation 6 ]$$

$${\hat{A}}_{m} = \frac{{\sum }_{i} {A}_{i}}{n} \qquad [ Equation 7 ]$$

In Equations 6 and 7, Aᵢ denotes the weight of the ith feature, Âₘ denotes the mean weight of biosignal m (e.g., Â_{EEG} denotes the mean weight of an EEG signal and Â_{ECG} denotes the mean weight of an ECG signal), and J denotes a bias. Other parameters may be substantially the same as those of Equations 3 to 5.

The training device 200, based on the first mean weight, the second mean weight, and the similarity, may adjust parameters of the neural network 400. The training device 200 may obtain a summed weight (hereinafter, also referred to as a combined weight) to balance contributions of the first feature and the second feature by summing the first mean weight and the second mean weight. A feature extracted from each of the biosignals 401 and 402 may include different types of information. The biosignals 401 and 402 may interact, and in a specific class classification, one of the biosignals 401 and 402 may contribute more significantly. To reflect such an interaction between the biosignals 401 and 402, the training device 200 may use the combined weight, obtained by summing the first mean weight and the second mean weight, for a loss function calculation. The combined weight may play a critical role in balancing contributions of the respective biosignals 401 and 402 in sentiment classification. The training device 200 may, for example, calculate the combined weight according to Equation 8 below.

$$\hat{A} = \frac{{\sum }_{n} {A}_{eeg} + {\sum }_{n} {A}_{ecg}}{n} = {\hat{A}}_{eeg} + {\hat{A}}_{ecg} \qquad [ Equation 8 ]$$

In Equation 8, Â denotes the combined weight, Â_{eeg} denotes the mean weight (e.g., the first mean weight) of the EEG signal 401, Â_{ecg} denotes the mean weight (e.g., the second mean weight) of the ECG signal 402, n denotes the total number of feature samples (i.e., the number of modality-aware attention networks), A_{eeg} denotes the weight of the EEG signal 401, and A_{ecg} denotes the weight of the ECG signal 402. Other parameters may be substantially the same as those of Equations 3 to 7.

Based on the summed weight and the similarity, the training device 200 may calculate a loss function (e.g., a proxy-based loss function) to adjust the balance between contributions of biosignals (e.g., the EEG signal 401 and the ECG signal 402) or features (e.g., the first feature 440 and the second feature 450). The loss function may be defined according to similarities between features and proxies for each of the biosignals 401 and 402 to ensure balanced training between the biosignals 401 and 402. The loss function may ensure that the same number of proxies is used for each of the biosignals 401 and 402 within a class and may uniformly adjust contributions of the biosignals 401 and 402, thereby achieving optimal performance in sentiment classification using the plurality of biosignals 401 and 402. For example, the loss function may enable all the biosignals 401 and 402 to contribute uniformly to sentiment classification and may be calculated as shown in Equation 9 below.

$${L}_{mami} = - log \frac{exp ( {S}_{i , y} + {A}_{y} + \text{?} )}{{\sum }_{c} exp ( {S}_{i , c} + {A}_{c} + \text{?} )} \qquad [ Equation 9 ]$$

In Equation 9, Lₘₐₘₗ denotes the loss function (e.g., a proxy-based loss function), S_{i,y} denotes the similarity normalized for class y, A_{y} denotes the attention weight of class y, and Â_{y} denotes the combined weight for the biosignals 401 and 402. Other parameters may be substantially the same as those of Equations 3 to 8.

The training device 200 may adjust parameters of the neural network 400 to minimize the loss function. When training of the neural network 400 is completed, the trained neural network may be driven to perform inference for sentiment classification.

FIG. 5 is a diagram illustrating the intra-modality attention network illustrated in FIG. 4.

Referring to FIG. 5, an intra-modality attention network 420 (e.g., the intra-modality attention network 420-1, 420-3, 420-5, or 420-7 of FIG. 4) may include a plurality of layers 510-1 to 510-N.

The plurality of layers 510-1 to 510-N may be formed in an encoder-decoder structure similar to U-Net. The layers 510-1 to 510-N may analyze extracted features in the multi-layer structure (e.g., U-Net). In other words, important information extracted from each layer 510-1 to 510-N may be emphasized by each layer, combined, and transmitted to a subsequent layer.

The plurality of layers 510-1 to 510-N may include a convolution layer (Conv), a batch normalization layer (Batch Norm), a rectified linear unit (ReLU) activation layer, and an attention layer 530.

The dimension of the convolution layer may be defined depending on the type of biosignal (e.g., the EEG signal 401 of FIG. 4 and the ECG signal 402 of FIG. 4). For example, in the intra-modality attention network 420-1 or 420-5 that processes the EEG signal 401, a convolution layer included in the layer 510-1 may be one-dimensional. In contrast, in the intra-modality attention network 420-3 or 420-7 that processes the ECG signal 402, the convolution layer included in the layer 510-1 may be two-dimensional. In other words, the plurality of layers 510-1 to 510-N may analyze a temporal pattern of the ECG signal 402 through a 1D-CNN and extract important features of the EEG signal 401 in a frequency band through a 2D-CNN.

A biosignal processed through a convolution layer may then be input to the attention layer 530 after undergoing batch normalization and ReLU activation.

The attention layer 530 may emphasize important features by calculating attention weights using a squeeze-and-excitation method and may suppress relatively less important features.

An attention weight calculated through the layer 510-1 may be transmitted to a subsequent layer 510-2. The results of the plurality of layers 510-1 to 510-N (e.g., attention weights calculated in each layer) may then be combined through hierarchical skip connections, and a final attention weight may be calculated.

FIG. 6 is a diagram illustrating the inter-modality attention network illustrated in FIG. 4.

Referring to FIG. 6, an inter-modality attention network 430 (e.g., the inter-modality attention network 430-1 or 430-3) may include a plurality of layers 610 to 690.

The plurality of layers may include max pooling layers 610-1 and 610-3, a concatenation layer 620 (also referred to as a connection layer), a convolution layer 630, a batch normalization layer 640, a ReLU activation layer 650, a split layer 660, convolution layers 670-1 and 670-3, sigmoid activation layers 680-1 and 680-3, and attention mask layers 690-1 and 690-3.

The plurality of layers may be classified into layers 610, 670, 680, and 690 that are used for each biosignal (e.g., the EEG signal 401 and the ECG signal 402) and layers 620, 630, 640, 650, and 660 that are commonly used for the biosignals.

The layers 620, 630, 640, 650, and 660 may particularly be used to analyze interactions between different biosignals and reflect the interactions in features (e.g., the first feature 440 (e.g., an EEG feature) of FIG. 4 and the second feature 450 (e.g., an ECG feature) of FIG. 4) of each biosignal.

The inter-modality attention network 430 may effectively model an interaction between the biosignals 401 and 402 and combine features extracted from the biosignals 401 and 402, thereby improving the performance of sentiment classification.

The features (e.g., the first feature 440 and the second feature 450) extracted from different biosignals 401 and 402 may be combined by considering an interaction between the biosignals 401 and 402, and important information may be emphasized through an attention mechanism.

The inter-modality attention network 430 may apply max pooling to the first feature 440 and the second feature 450, which are extracted from different biosignals 401 and 402, respectively, through max pooling layers 610-1 and 610-3, and may combine the features through the concatenation layer 620. The inter-modality attention network 430 may model an interaction between the biosignals 401 and 402 by processing the combined feature through the convolution layer 630. The inter-modality attention network 430 may evaluate the importance of each of the biosignals 401 and 402 in the interaction through the batch normalization layer 640 and the ReLU activation layer 650. After the evaluation, the inter-modality attention network 430 may separate the features according to the biosignals 401 and 402 through the split layer 660. Attention masks corresponding to the first feature 440 and the second feature 450 may be generated from the separated features through layers 670-1, 680-1, and 690-1, and layers 670-3, 680-3, and 690-3, respectively. By applying the attention masks corresponding to the first feature 440 and the second feature 450, the first feature 440 and the second feature 450 may be adjusted. The adjusted first feature 440 and second feature 450 may emphasize important features and suppress less important features in sentiment classification, and may be combined and transmitted to a subsequent network (e.g., a subsequent modality-aware attention network or the modality-aware attention network 410-3 of FIG. 4) and/or a classifier (e.g., the M1 classifier 460 and/or the M2 classifier 470 of FIG. 4).

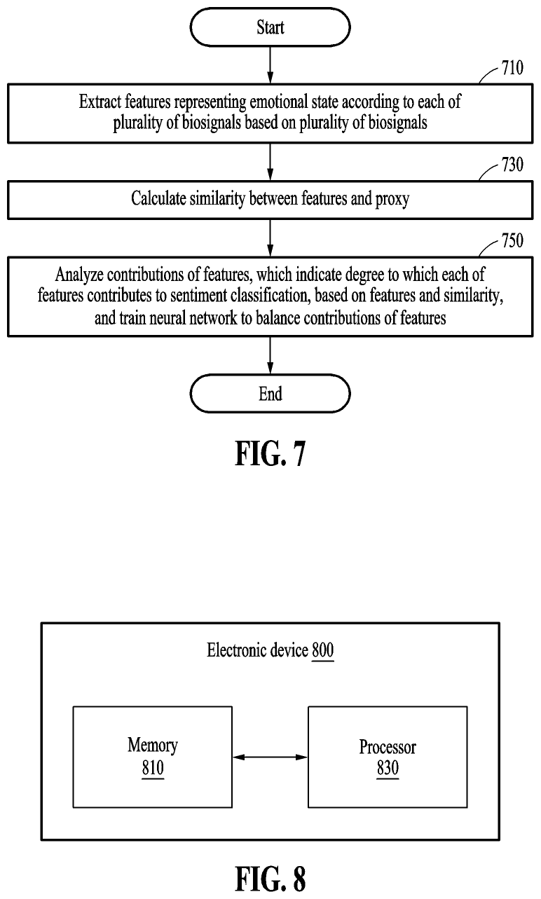

FIG. 7 is a flowchart illustrating a training method of a neural network according to an embodiment.

Operations 710 to 750 may be sequentially performed, but the present disclosure is not limited thereto. For example, two or more operations may be performed in parallel. Operations 710 to 750 may be substantially the same as the operations of the training device (e.g., the training device 200 of FIG. 2B) described with reference to FIGS. 1 to 6. Accordingly, a further description thereof is not repeated herein.

In operation 710, the training device 200 may extract features representing an emotional state according to each of a plurality of biosignals based on the plurality of biosignals. The plurality of biosignals may include an EEG signal and an ECG signal. The features may include a first feature corresponding to the EEG signal and a second feature corresponding to the ECG signal.

In operation 730, the training device 200 may calculate a similarity between the features and a proxy. The proxy may include representative features of a class that serves as a criterion for sentiment classification. The class may include an arousal class and a valence class and may be a category according to a binary sentiment classification.

In operation 750, the training device 200 may analyze contributions of the features, which indicate a degree to which each of the features contributes to sentiment classification, based on the features and the similarity, and may train the neural network to balance the contributions of the features.

FIG. 8 illustrates an example of an electronic device according to an embodiment.

Referring to FIG. 8, an electronic device 800 may include a memory 810 and a processor 830. The description provided with reference to FIGS. 1 to 7 may also apply to FIG. 8. For example, the training device 200 of FIG. 2B may be the electronic device 800.

The memory 810 may store instructions (or programs) executable by the processor 830. For example, the instructions include instructions for executing the operations of the processor 830 and/or operations of each component of the processor 830.

The memory 810 may be implemented as a volatile memory device or a non-volatile memory device.

The volatile memory device may be implemented as a dynamic random-access memory (DRAM), a static RAM (SRAM), a thyristor RAM (T-RAM), a zero capacitor RAM (Z-RAM), or a twin transistor RAM (TTRAM).

The non-volatile memory device may be implemented as an electrically erasable programmable read-only memory (EEPROM), a flash memory, a magnetic RAM (MRAM), a spin-transfer torque (STT)-MRAM, a conductive bridging RAM (CBRAM), a ferroelectric RAM (FeRAM), a phase change RAM (PRAM), a resistive RAM (RRAM), a nanotube RRAM, a polymer RAM (PoRAM), a nano floating gate Memory (NFGM), a holographic memory, a molecular electronic memory device), or an insulator resistance change memory.

The processor 830 may process data stored in the memory 810. The processor 830 may execute computer-readable code (e.g., software) stored in the memory 810 and instructions triggered by the processor 830.

The processor 830 may be a data processing device implemented by hardware including a circuit having a physical structure to perform desired operations. For example, the desired operations may include code or instructions included in a program.

The hardware-implemented data processing device may include, for example, a microprocessor, a central processing unit (CPU), a processor core, a multi-core processor, a multiprocessor, an application-specific integrated circuit (ASIC), and a field-programmable gate array (FPGA).

The processor 830 may cause the electronic device 800 to perform one or more operations by executing the instructions and/or code stored in the memory 810. The operations performed by the electronic device 800 may be substantially the same as the operations performed by the training device 200 described above with reference to FIGS. 2A to 7. Accordingly, the repeated description thereof is omitted.

The examples described herein may be implemented by using a hardware component, a software component, and/or a combination thereof. For example, the devices, the methods, and the components described in the embodiments may be implemented using a general-purpose or special-purpose computer, such as a processor, a controller and an arithmetic logic unit (ALU), a digital signal processor (DSP), a microcomputer, a FPGA, a programmable logic unit (PLU), a microprocessor, or any other devices capable of responding to and executing instructions. The processing device may run an operating system (OS) and one or more software applications that run on the OS. The processing unit also may access, store, manipulate, process, and generate data in response to execution of the software. For purpose of simplicity, the description of a processing unit is used as singular; however, one skilled in the art will appreciate that a processing unit may include a plurality of processing elements and a plurality of types of processing elements. For example, the processing unit may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and/or DVDs; magneto-optical media such as optical discs; and hardware devices that are specially configured to store and perform program instructions, such as read-only memory (ROM), random-access memory (RAM), flash memory, and the like. Examples of program instructions include both machine code, such as produced by a compiler, and files containing higher-level code that may be executed by the computer using an interpreter.

The above-described hardware devices may be configured to act as one or more software modules in order to perform the operations of the above-described examples, or vice versa.

As described above, although the examples have been described with reference to the limited drawings, a person skilled in the art may apply various technical modifications and variations based thereon. Suitable results may be achieved when the described techniques are performed in a different order, and/or if components in a described system, architecture, device, or circuit are combined in a different manner, and/or replaced or supplemented by other components or their equivalents.

Accordingly, other implementations are within the scope of the following claims.

What is claimed is:

1. A method of training a neural network to perform sentiment classification, the method comprising:
extracting, based on a plurality of biosignals, features representing an emotional state according to each of the plurality of biosignals;
calculating a similarity between the features and a proxy; and
analyzing, based on the features and the similarity, contributions indicating a degree to which each of the features contributes to the sentiment classification, and training the neural network to balance the contributions of the features;
wherein the proxy comprises representative features of a class that serves as a criterion for the sentiment classification.

2. The method of claim 1, wherein the class comprises an arousal class and a valence class.

3. The method of claim 1, wherein the plurality of biosignals comprises an electroencephalography (EEG) signal and an electrocardiography (ECG) signal.

4. The method of claim 3, wherein the features comprise a first feature corresponding to the EEG signal and a second feature corresponding to the ECG signal.

5. The method of claim 4, wherein the training of the neural network comprises:
calculating, based on the similarity, an attention weight to adjust contributions of the first feature and the second feature;
calculating, based on the attention weight, the proxy, and the first feature, a first mean weight based on the first feature;
calculating, based on the attention weight, the proxy, and the second feature, a second mean weight based on the second feature; and
adjusting, based on the first mean weight, the second mean weight, and the similarity, parameters of the neural network.

6. The method of claim 5, wherein the adjusting of the parameters of the neural network comprises:
summing the first mean weight and the second mean weight to obtain a summed weight for balancing a contribution of the first feature and a contribution of the second feature; and
calculating, based on the summed weight and the similarity, a loss function for adjusting a balance between the contributions.

7. The method of claim 6, wherein the adjusting of the parameters of the neural network comprises adjusting the parameters of the neural network so that the loss function is minimized.

8. An electronic device for training a neural network to perform sentiment classification, the electronic device comprising:
a processor; and
a memory configured to store instructions,
wherein the instructions, when executed individually or collectively by the processor, cause the electronic device to:
extract, based on a plurality of biosignals, features representing an emotional state according to each of the plurality of biosignals;
calculate a similarity between the features and a proxy; and
analyze, based on the features and the similarity, contributions indicating a degree to which each of the features contributes to the sentiment classification, and train the neural network to balance the contributions of the features;
wherein the proxy comprises representative features of a class that serves as a criterion for sentiment classification.

9. The electronic device of claim 8, wherein the class comprises an arousal class and a valence class.

10. The electronic device of claim 8, wherein the plurality of biosignals comprises an electroencephalography (EEG) signal and an electrocardiography (ECG) signal.

11. The electronic device of claim 10, wherein the features comprise a first feature corresponding to the EEG signal and a second feature corresponding to the ECG signal.

12. The electronic device of claim 11, wherein the instructions, when executed individually or collectively by the processor, cause the electronic device to:
calculate, based on the similarity, an attention weight to adjust contributions of the first feature and the second feature;
calculate, based on the attention weight, the proxy, and the first feature, a first mean weight based on the first feature;
calculate, based on the attention weight, the proxy, and the second feature, a second mean weight based on the second feature; and
adjust, based on the first mean weight, the second mean weight, and the similarity, parameters of the neural network.

13. The electronic device of claim 12, wherein the instructions, when executed individually or collectively by the processor, cause the electronic device to:
sum the first mean weight and the second mean weight to obtain a summed weight for balancing a contribution of the first feature and a contribution of the second feature; and
calculate, based on the summed weight and the similarity, a loss function for adjusting a balance between the contributions.

14. The electronic device of claim 13, wherein the instructions, when executed individually or collectively by the processor, cause the electronic device to:
adjust the parameters of the neural network so that the loss function is minimized.
